(19) United States
(12) Patent Application Publication
(10) Pub. No.: US 2005/0032698 A1
(43) Pub. Date: Feb. 10, 2005

(54) Stabilized formulation of parathyroid hormone

(75) Inventors: David Day, Salt Lake City, UT (US); Ananda Seneviratne, Mississauga (CA); Karen Toney, Mississauga (CA)

Correspondence Address: FOLEY AND LARDNER;SUITE 500, 3000 K STREET NW, WASHINGTON, DC, 20007, US

(73) Assignees: NPS Allelix Corp.; NPS Pharmaceuticals, Inc.

(21) Appl. No.: 10/890,520

(22) Filed: Jul. 14, 2004

Related U.S. Application Data
(60) Provisional application No. 60/486,464, filed on Jul. 14, 2003.

(51) Int. Cl. A61K 38/29

(52) U.S. Cl. 514/12

(57) ABSTRACT

The invention relates to PTH formulations in which the PTH is present with at least one divalent cation effective to stabilize the PTH.

This is a U.S. regular application that claims priority to U.S. provisional application Ser. No. 60/486,464, filed on Jul. 14, 2003, and which is incorporated herein by reference.

### FIELD OF THE INVENTION

This invention relates to pharmaceutical formulations. More particularly, the invention relates to stable parathyroid hormone formulations, to their production, and to their use for instance in the treatment of patients suffering from osteoporosis.

### BACKGROUND TO THE INVENTION

Parathyroid hormone (PTH) is a protein secreted from the mammalian parathyroid gland, and exerts a variety of effects. Its role in calcium homeostasis has prompted a wealth of research that has culminated in the clinical use of parathyroid hormone and certain active fragments as a treatment for various bone disorders, particularly including osteoporosis.

Typically, PTH is made available as a lyophilized powder that is then reconstituted in an aqueous vehicle either by the end-user or the attending physician. A variety of such powdered PTH preparations have been proposed, for example in U.S. Pat. No. 5,495,801 and in U.S. Pat. No. 5,563,122.

For the convenience of the end-user, PTH has also been prepared in multidose formulations that provide for multiple sequential injections to be administered from a single liquid preparation, as described for instance in WO 99/055353. To allow for this, the PTH must not only be formulated at relative high PTH concentration, but must also remain stable in liquid for multiple days over which the multidose formulation will be stored during use.

Regardless of the form in which, or the concentration at which, the PTH must be formulated, the challenge in formulating PTH lies in providing a composition in which the PTH remains stable under the intended storage conditions. PTH is particularly demanding in this respect. The loss of N-terminal residues, re-arrangement or modification of internal residues, and the loss of a major C-terminal portion all contribute to significant loss of biological activity. Moreover, PTH tends to aggregate and precipitate from solution particularly when formulated at high concentration.

It is, accordingly, an object of the present invention to provide a stable parathyroid hormone formulation.

It is another object of the present invention to provide a stable parathyroid hormone formulation that is useful as a multi-dose formulation.

### SUMMARY OF THE INVENTION

It has now been found that divalent cations improve the stability of parathyroid hormone in solution. In one of its aspects therefore, the present invention provides a formulation comprising parathyroid hormone and a divalent cation in an amount effective to stabilize the parathyroid hormone.

In one aspect of the invention, an injectable formulation comprising parathyroid hormone, an aqueous vehicle, and at least one divalent cation in an amount effective to stabilize the parathyroid hormone is provided.

In one embodiment, the divalent cation is cationic magnesium, zinc, or a combination thereof. In a preferred embodiment, the divalent cation is present as a zinc chloride salt.

In one embodiment, the divalent cation is present at a concentration within the range from about 10 to about 50 mM. Preferably, the divalent cation is present at a concentration within the range from about 20 to about 40 mM

In another embodiment, the PTH is present in a unit dose. In yet another embodiment, the PTH is present in amount effective for administration of more than one unit dose.

In yet another aspect of the present invention, a lyophilized formulation is provided which, upon reconstitution in an aqueous vehicle, produces the above-described injectable formulation.

A further aspect of the present invention provides a device for use in delivering a drug by injection, the device comprising a lyophilized formulation of the injectable formulation described herein, and an aqueous vehicle for reconstituting the lyophilized formulation.

In another aspect of the present invention, a PTH formulation is provided that comprises an aqueous vehicle, PTH at a concentration within the range from about 1 mg/mL to about 2 mg/mL and zinc chloride at a concentration in the range from about 20 to about 40 mM.

The present invention also provides a process for preparing a formulation comprising stabilized PTH, comprising the step of combining PTH, an amount of at least one divalent cation effective to stabilize said PTH, and water.

A process for preparing an injectable formulation comprising stabilized PTH is provided which comprises the step of reconstituting, in an aqueous vehicle, a lyophilized preparation comprising PTH and at least one divalent cation in an amount effective to stabilize the reconstituted PTH.

In yet another embodiment, another process for preparing an injectable formulation comprising stabilized PTH is provided, comprising the step of reconstituting, in an aqueous vehicle, a lyophilized preparation comprising PTH, wherein said vehicle comprises at least one divalent cation in an amount effective to stabilize the reconstituted PTH.

The present invention also provides a method of treating osteoporosis comprising the steps of administering to a subject in need thereof any formulation described herein. In one embodiment, the subject is a human.

In yet another aspect of the invention, a method of diagnosing hypoparathyroidism is provided, which comprises the steps of
(a) administering to a subject in need thereof a PTH formulation according to claim 1; and
(b) determining the effect of the administered PTH on urinary cAMP levels, with cAMP elevation being indicative of a hypoparathyroid condition.

In one embodiment, the subject is a human.

In certain embodiments of the invention, the formulation is provided as a solution, or as a lyophilized powder from which a solution is generated by reconstitution with a suitable liquid vehicle. In preferred embodiments, the formulation is adapted for delivery by injection.

More details about these embodiments of the invention are now described with reference to the accompanying drawings.

### BRIEF REFERENCE TO THE DRAWINGS

FIG. 1 is a graph showing the effect of various divalent cations on PTH stability;

FIG. 2 is a graph showing the stability of a given PTH formulation over time at various temperatures; and

FIG. 3 is a graph showing stability of different PTH formulations under various conditions.

Both the foregoing general description and brief reference to the drawings, and the following detailed description, are exemplary and explanatory and are intended to provide further explanation of the invention as claimed. Other objects, advantages, and novel features will be readily apparent to those skilled in the art from the following detailed description of the invention. All of the publications cited here are incorporated herein by reference.

### DETAILED DESCRIPTION OF THE INVENTION AND PREFERRED EMBODIMENTS

The invention relates to PTH formulations in which the PTH is present with at least one divalent cation effective to stabilize the PTH.

A. Scope of the Term “Parathyroid Hormone”

As used herein, the terms “parathyroid hormone” and “PTH” are used interchangeably and encompass: (1) human parathyroid hormone (hPTH; hPTH(1-84)), which is an 84 amino acid protein having the sequence reported by Kimura et al. in Biochem. Biophys. Res. Comm., “Solution Synthesis of (ASN76)— human parathyroid hormone (1-84)”, 114(2):493-499 (1983); (2) vertebrate homologs thereof, including bovine PTH, porcine PTH and the like; (3) bioactive fragments thereof, and (4) bioactive analogs of such proteins and their bioactive fragments.

As used herein, analogs and fragments are considered bioactive if they stimulate adenylate cyclase when incubated appropriately with a reporter cell line such as UMR-106. An assay suitable for making this determination is well established in the PTH literature and is described for instance by Rodan et al. in J. Clin. Invest., 72:1511 (1983), and by Rabbani et al. in Endocrinol., 123:2709 (1988). Particularly preferred PTH compounds are those which exhibit human PTH activity when assessed using the ovariectomized rat model of osteoporosis reported by Kimmel et al., Endocrinology, 32(4):1577 (1993).

Vertebrate homologs of hPTH that can be formulated as herein described include mammalian homologs of HPTH, including bovine PTH and porcine PTH having the amino acid sequences reported for instance by Keutmann et al., Current Research on Calcium Regulating Hormones, Cooper, C. W. (Ed), PP. 57-63 (University of Texas Press, Austin, 1987).

Bioactive fragments of full length hPTH(1-84) or vertebrate homologs thereof are those peptides that retain PTH activity and are an N-terminal fragment of the full length PTH. Preferably, at least the first 27 amino acids of PTH, and more desirably at least the first 34 amino acids of PTH are present. For instance, PTH fragments that can be formulated in accordance with the present invention include PTH(1-31), PTH(1-34), PTH(1-37), PTH(1-38), and PTH(1-41).

Analogs of PTH or a PTH fragment retain PTH activity and incorporate at least one and up to about 5 or more amino acid substitutions, additions, or internal deletions and combinations thereof. Such analogs include for instance, those in which one or more resident methionine residues are substituted by an oxidatively more stable amino acid, such as Leu or Ile, or by a class-distinct residue such as Cys; and those in which one or more of the residues sensitive to enzymatic attack, e.g., at positions 25-27, are substituted or modified to inhibit digestion thereat.

B. Preferred Dosages of PTH

Desirably, the PTH in the formulation is present in an amount useful medically in the treatment of a bone disorder. Such amounts include those that result in the administration of PTH in a unit dose by single injection, as well as those amounts useful in a single formulation to deliver multiple consecutive doses to the end-user. In one embodiment, the PTH formulations are formulated to provide a unit dose of PTH, i.e., as single dose formulations. These unit dose formulations desirably comprises from about 20 μg to about 150 μg of PTH, more preferably about 50 μg to about 150 μg of PTH. In another embodiment, the PTH formulations are formulated to provide for the administration of multiple doses from a single formulation. Such multidose formulations desirably incorporate from about two (2) to about twenty-eight (28) unit doses of PTH, e.g., 7, 14, 21, or 28 unit doses of PTH, and thus incorporate PTH at a concentration that is the corresponding multiple of the PTH unit dose. Thus, for example, for multidose administration, the present PTH formulations can contain from about 150 μg PTH/mL of formulation up to about 5 mg of PTH/mL of formulation. In a specific embodiment, the PTH formulation comprises 14 unit doses of hPTH(1-84), e.g., about 1.2 to about 1.8 mgs/mL of PTH.

C. Divalent Cations Useful in the Invention

The divalent cation useful in the formulation can be any divalent cation, and is suitably selected from cationic calcium and, more preferably magnesium, and most preferably zinc. The divalent cation is desirably supplied as a water soluble salt, suitably of an organic or inorganic acid, most suitably as the chloride. Thus, the present formulations are preferably those in which the parathyroid hormone is stabilized using the chloride salt of calcium, magnesium, or most preferably zinc, or combinations thereof.

The desired amount of divalent cation present in the formulation is an amount that stabilizes the parathyroid hormone, as determined for instance using the assay herein reported. In this assay, the measured end-point is the time in days taken for a visible precipitate to form, under the noted assay conditions. Preferred concentrations of cation are those which prolong the formation of precipitants, relative to a suitable control.

In a preferred embodiment, in which the PTH is present at a concentration of about 1.4 mg/mL, the concentration of cation present in the PTH formulation lies within the range from about 15 mM cation to about 50 mM cation. In the case of Zn cation, the preferred concentration is about 20 to about 40 mM Zn, e.g., about 30 mM Zn, as ZnCl₂.

D. Exemplary PTH Dosage Forms of the Invention

The PTH formulation is desirably adapted for administration by injection, and is accordingly formulated in a pharmaceutically acceptable, aqueous vehicle. The present formulations thus desirably further comprise an aqueous vehicle that is optionally buffered to a pH in the range from about 3.0 to about 7.0. Preferably, the pH is adjusted to lie within the range from about 4.0 to about 6.5, more preferably from about 4.5 to about 6.0. Suitable buffering agents include citrate, acetate, and phosphate.

In the case of multidose formulations to be administered as a liquid, the PTH formulations desirably further comprise a preservative useful to control the growth of microflora. Suitable preservatives include benzyl alcohol and, preferably, m-cresol.

The present PTH formulations may further comprise other suitable excipients and ingredients as are common in the parenteral formulation art, including sodium chloride, stabilizing agents such dextran sulfate or related polyanion, and the like. It is to be understood that water-insoluble components such as lipids and phospholipids desirably are avoided so that a generally homogeneous solution can result.

Whether or not the PTH is formulated for unit or multiple dosing, the PTH formulation can be provided as a lyophilized preparation, for reconstitution using an appropriate aqueous vehicle, or as a liquid preparation. For lyophilization, the PTH formulations suitably incorporate a bulking agent that protects the PTH during the freeze-drying process and promotes formation of a cake of desired quality. Suitable bulking agents include the various sugars and polyols such as maltose, lactose, and the like. A preferred bulking agent is mannitol. Lyophilized PTH formulations are prepared by first preparing the liquid formulation in bulk, comprising the PTH, bulking agent, and buffering agent, and any other desired ingredients and then subjecting aliquots thereof, usually in ampoules, vials, or carpules, to the standard freeze-drying process. The divalent cation can be provided in the PTH formulation that is lyophilized, or the divalent cation can be provided in the solvent used to reconstitute the lyophilized PTH preparation.

In lyophilization, or freeze-drying, the process entails a temperature cycling process that is controlled carefully to ensure that drying proceeds to substantial completion, i.e., to yield a powder containing not more than about 6% water by weight, more preferably not more than about 2 to about 3% water by weight. A protocol suitable for obtaining the freeze-dried formulations of PTH entails subjecting vials or carpules filled with aqueous PTH preparation (with cation or without cation if provided instead in the reconstitution vehicle) to a drying process having generally two different drying stages, the first being performed to drive unbound water from the aqueous preparation without causing collapse of the cake. This can be achieved by first cooling the vialed aqueous PTH preparation to a product-ice temperature of lower than −30° C., more preferably about −50° C. or lower, and then increasing shelf temperature to, and holding at, about −10° C., under reduced pressure of not more than about 350 μbar, e.g., about 260 μbar, until substantially all unbound water is driven off. A drying time of about 16 hours may be appropriate. The second drying cycle is designed to liberate bound water from the cake without causing collapse and using a temperature below that deleterious to the PTH bioactivity. This second drying step can be performed under further reduced pressure (e.g., about <50 μbar) at about −10° C. for about 3 hours, then warming to and holding at about 25° C. until substantially all of the bound water is driven off, e.g., for about 12-16 hours or more. On completion, the vials or carpules can be sealed, for example, by automated stoppering or fusing, and then removed from the freeze-drier and capped.

For unit dose formulations, the lyophilized PTH formulation can be reconstituted in an aqueous vehicle for immediate use. For multiple dose preparations, and in the case where a preservative is desired, the PTH can either be lyophilized from a solution to which the preservative has been added or, more preferably, the preservative can be introduced to the lyophilized PTH by reconstitution using water supplemented with the preservative. As noted about, the aqueous vehicle can further comprise the divalent cation if not present in the lyophilized PTH preparation.

Thus, the present invention further contemplates lyophilized forms of the injectable, aqueous PTH preparations. Suitably, such lyophilized preparations comprises less than about 6% water by weight, more preferably less than about 3% water by weight, and even more preferably less than about 2% water by weight. For lyophilization, the formulation also suitably contains a bulking agent such as mannitol.

In a preferred embodiment, there is provided a formulation for injection, comprising parathyroid hormone, an amount of divalent cation effective to stabilize the parathyroid hormone, and a pharmaceutically acceptable, aqueous vehicle.

In another preferred embodiment, there is provided a pharmaceutical combination useful to prepare a formulation for injection. The combination comprises a lyophilized preparation comprising parathyroid hormone and an amount of divalent cation effective to stabilize the parathyroid hormone and, for reconstituting the lyophilized preparation, an aqueous vehicle. If injection is to be done with a powder formulation, re-constitution of the lyophilized preparation in an aqueous vehicle is not required.

For example, in use, the preparation is provided as a powder that is reconstituted at the time of administration using the aqueous vehicle which may be supplied in a separate container. In one embodiment, the formulation is provided in a form adapted for use in an injection pen, in which the PTH formulation is provided in one chamber of a dual chamber cartridge as a lyophilized powder which dual chamber cartridge is loaded into the injection pen. When the injection pen is actuated, the PTH lyophilized powder is mixed with the aqueous vehicle in the second chamber of the dual chamber cartridge to form a liquid PTH formulation.

E. Methods of Using the PTH Formulations of the Invention

The formulation of the present invention can be used in the treatment of patients afflicted with various bone disorders including osteoporosis. The treatment regimen will be determined by an attending physician, but may entail once daily, subcutaneous injection of a unit dose of PTH each day for a treatment period which could extend from a period of several weeks to many months. The PTH can be used alone, or in combination with other drugs useful in the treatment of such diseases.

Moreover, the present formulation can be used for diagnostic purposes in relation, for instance, to hypoparathyroidism and pseudohypoparathyroidism. Appropriate for this purpose is an intravenously infused unit, equal to 200 International units of hPTH(1-84) or of a homolog or an analog thereof. Diagnosis is made by determining the effect of the administered dose on urinary cAMP levels, with cAMP elevation being indicative of the hypoparathyroid condition rather than of its pseudoform.

To elaborate, PTH is typically missing in patients with hypoparathyroidism, whereas patients diagnosed with pseudohypoparathyroidism are unable to respond to PTH. See Mallette, Ann. Intern. Med., 109(10): 800-4, 1988. Accordingly, a diagnose of hypoparathyroidism or pseudohypoparathyroidism can derive from monitoring an patient's reaction to exogenous PTH.

In this respect, Kruse and Kracht, Eur. J. Pediatr., 146(4): 373-7, 1987, describes a test to distinguish hypoparathyroidism from pseudohypoparathyroidism by measuring plasma cAMP levels in a patient, at 5 and 10 minutes after the patient is injected with a PTH fragment, 1-38 hPTH.

The protocol suggested by Mallette (1988), supra, entails treating patients with a different PTH fragment, 1-34 PTH (“teriparatide”). The method requires measuring phosphate and creatinine levels, in serum and urine, before and after teriparatide administration, and was found to distinguish between Type 1 and Type 2 pseudohypoparathyroidism. That is, a “blunted” cAMP response to teriparatide indicates Type 1, while patients with Type 2 produce “normal” cAMP responses.

In Mallete's study, both Type 1 and Type 2 pseudohypoparathyroidism were found to exhibit high levels of baseline immunoreactive PTH, as opposed to low levels found in patients with idiopathic hypoparathyroidism. Accordingly, Mallette's methodology also can distinguish Type 1 and Type 2 pseudohypoparathyroidism from hypoparathyroidism. Mallette provides formulae for calculating such response values in Table 3 at page 802 of that publication.

To complement the diagnostic uses of PTH, Winer et al., JAMA., 276(8): 631-6, 1996, discovered that patients with hypoparathyroidism, who had received “PTH 1-34,” had reduced levels of calcium excreted in their urine, compared to calcium leveles excreted by patients who were given vitamin D-based treatments like calcitriol. Accordingly, Winer et al. concluded that administration of a PTH 1-34 fragment reduced the risk of hypercalciuria in those patients.

In a subsequent study, Winer et al., J. Clin. Endocrinol. Metab., 83(10): 3480-6, 1998, devised a “twice-daily” PTH administration regimen that allowed for a marked reduction in the total daily PTH dose. Furthermore, that regimen resulted in “less fluctuation in serum calcium, normalization of urine calcium, and significantly improved metabolic control in patients with calcium receptor mutations” (page 3480, second column). Winer et al. (1998) also reported a decreased incidence of bone pain, compared to a “once-only” dosage regimen.

By the same token, the PTH proteins and protein fragments of the present invention may be used diagnostic and therapeutic purposes, along the foregoing lines. That is, the cation-complexed PTH described here may be employed, for instance, to (a) differentiate hypoparathyroidism from pseudohypoparathyroidism, (b) differentiate Type 1 pseudohypoparathyroidism from Type 2 pseudohypoparathyroidism, (c) reduce the risk of hypercalciuria, especially in patients with hypoparathyroidism, (d) normalize calcium levels, and (e) improve the quality of life by reducing pain associated with such disorders.

The present invention is described herein using several definitions, as set forth below and throughout the application.

“About” will be understood by persons of ordinary skill in the art and will vary to some extent on the context in which the term is used. If there are uses of the term which are not clear to persons of ordinary skill in the art given the context in which it is used, “about” will mean up to plus or minus 10% of the particular term.

“Therapeutically effective amount” as used herein with respect to a PTH dosage shall mean a dosage that provides the specific pharmacological response for which the PTH is administered in a significant number of subjects in need of such treatment. It is emphasized that “therapeutically effective amount,” administered to a particular subject in a particular instance will not always be effective in treating the diseases described herein, even though such dosage is deemed a ‘therapeutically effective amount’ by those skilled in the art. It is to be further understood that PTH dosages are, in particular instances, measured as oral dosages, or with reference to PTH levels as measured in blood.

The following example is given to illustrate the present invention. It should be understood, however, that the invention is not to be limited to the specific conditions or details described in this example. Throughout the specification, any and all references to a publicly available document, including a U.S. patent, are specifically incorporated by reference.

### EXAMPLE 1

Studies showed that divalent cations such as Zn²⁺, Mg²⁺, and Ca²⁺ improve the stability of PTH. The most preferred cation for hPTH (1-84) is zinc chloride (ZnCl₂, USP grade, CAS# 7646-85-7, FW 136.3). Various concentrations of ZnCl₂ were tested. A preferred concentration of 30 mM (0.4%, w/v) in the final reconstituted PTH formulation was identified. The results of ZnCl₂ studies are given below.

In these experiments, the divalent cation was added as a reconstitution solution to a lyophilized preparation contained in a carpule formed from a batch PTH formulation having the ingredients noted below. The final liquid PTH formulation, following reconstitution, contained hPTH(1-84) at a final concentration of 1.4 mg/ml in 1.15 mL total volume.

Dual-chamber carpules containing a lyophilized PTH formulation were prepared from a batch PTH formulation. The components are approximately as follows:

Chamber 1: Composition of lyophilized PTH

| | |
|---|---|
| hPTH(1-84) | 1.68 mg |
| citric acid monohydrate | 1.26 mg |
| mannitol | 30 mg |
| NaCl | 3.9-4.6 mg |
| NaOH | pH 5.5(qs) |
| HCl | pH 5.5(qs) |

Chamber 2: For reconstitution

| | |
|---|---|
| m-cresol | 3.2 mg/mL |
| water | 1.13 mL |
| Divalent cation | as tested |

On reconstitution, each carpule contained approximately 1.4 mg/mL PTH with a total volume of approximately 1.15 mL.

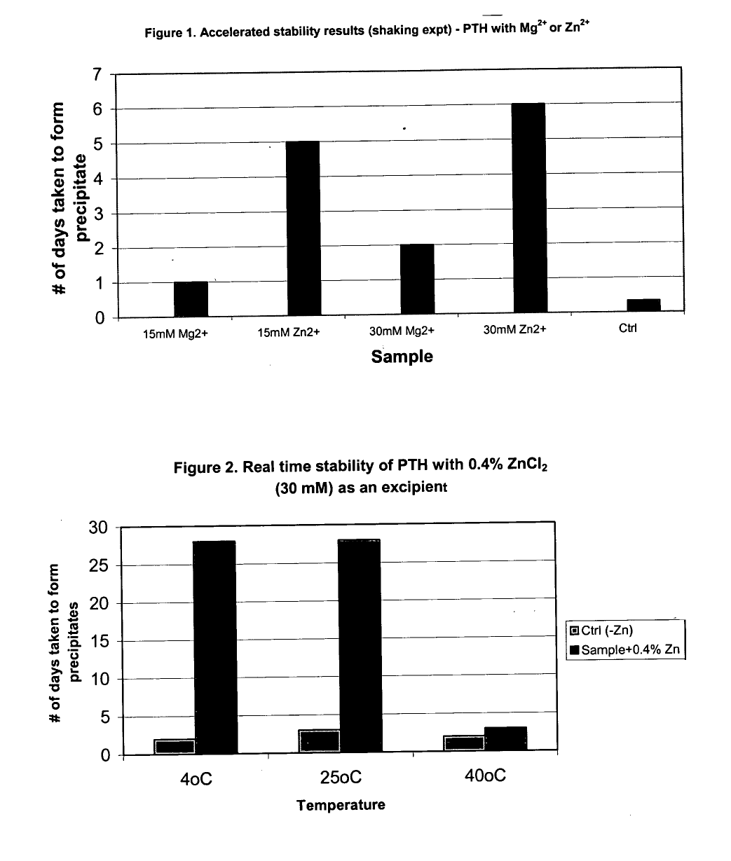

The following results in FIG. 1 are based on a shaking stress test, and the PTH formulation (as formulated above) with the addition of either MgCl₂ or ZnCl₂. The shaking stress was introduced by placing carpules on a Thermoline mixer where they were subjected to repetitive and gentle inversions, at room temperature. The results showed 30 mM ZnCl₂ is the best stabilizer among those tested.

FIG. 2 shows the real time stability results at various temperatures after reconstitution of PTH (formulated as above) in a given carpule with a reconstitution solvent that contains 30 mM ZnCl₂ as a stabilizer. Control samples without ZnCl₂ show only 2-3 days stability at all three storage conditions. In surprising contrast, samples with ZnCl₂ were stable for 28 days at 4° C. and room temperature.

Results of the bioassay (PTH-adenylate cyclase assay) of the Zn and Mg formulated samples for the biological activities are given in Table 1. The adenylate cyclase bioassay is based on PTH-stimulated adenylate cyclase activity in the rat osteosarcoma cell line UMR-106. Tritiated adenine is metabolically incorporated into ATP, and adenylate cyclase stimulated by PTH converts the tritiated ATP to tritiated cAMP. The tritiated cAMP is separated from tritiated ATP by a two step column process. The column samples also contain C-14-cAMP as an internal standard that is used to calculate column recoveries. Fully active PTH is characterized by a Kd in this assay of between 0.1 and 1.2 nM. As shown by the data, PTH samples containing either Zn or Mg cations remain biologically active.

TABLE 1

| Sample Type | PTH + 30 mM Zn²⁺ | PTH + 30 mM Mg²⁺ | Ctrl |
|---|---|---|---|
| Mean Kd (nM) | 0.54 | 0.6 | 0.52 |
| % RSD | 13 | 14 | 41 |

Other formulations of hPTH(1-84) were also produced, as follows:

| PTH | 1.4 mg/mL |
|---|---|
| Citrate | 1.1 mg/mL |
| Mannitol | 26 mg/mL |
| NaCl | 2.7 mg/mL |
| ZnCl₂ | 4.0 mg/mL |
| m-cresol | 3.2 mg/mL |
| pH | 5.5 |

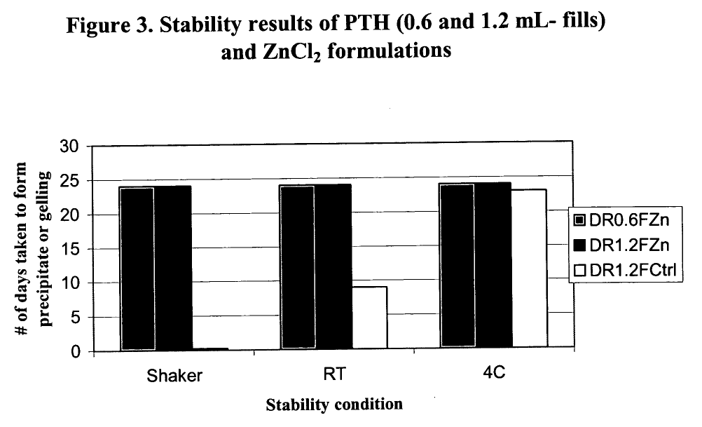

Samples of this formulation, in 0.6 mL and 1.2 mL volumes, were also subjected to stability trials. Results are shown in FIG. 3.

As noted, the presence of Zn cation significantly enhanced stability of the PTH, by prolonging the period before either precipitation or gelling occurred.

In a like manner, formulations of hPTH(1-34) can be prepared as a 3.3 mL, unit dose solution containing 0.25 mg hPTH(1-34), 0.41 mg glacial acetic acid, 0.10 mg sodium acetate (anhydrous), 45.4 mg mannitol, 3.0 mg m-cresol, a divalent cation such as ZnCl₂ at the desired concentration in an amount to further stabilize such PTH, e.g. 30 mM, and water for injection.

It will thus be appreciated that PTH formulated in a solution containing divalent cation enhances the stability of the formulated PTH, and particularly retards and/or delays PTH precipitation and the formation of a gelled solution.

It will be apparent to those skilled in the art that various modifications and variations can be made in the methods and compositions of the present invention without departing from the spirit and scope of the invention. Thus, it is intended that the present invention cover the modifications and variations provided they come within the scope of the appended claims and their equivalents.

1. An injectable formulation comprising parathyroid hormone, an aqueous vehicle, and at least one divalent cation in an amount effective to stabilize the parathyroid hormone.

2. The injectable formulation according to claim 1, wherein the divalent cation is cationic magnesium, zinc, or a combination thereof.

3. The injectable formulation according to claim 1, wherein the divalent cation is present as a zinc chloride salt.

4. The injectable formulation according to claim 1, wherein the divalent cation is present at a concentration within the range from about 10 to about 50 mM.

5. The injectable formulation according to claim 4, wherein the divalent cation is present at a concentration within the range from about 20 to about 40 mM

6. The injectable formulation according to claim 1, wherein the PTH is present in a unit dose.

7. The injectable formulation according to claim 1, wherein the PTH is present in amount effective for administration of more than one unit dose.

8. A lyophilized formulation which, upon reconstitution in an aqueous vehicle, produces an injectable formulation according to claim 1.

9. A device for use in delivering a drug by injection, the device comprising a lyophilized formulation of the injectable formulation according to claim 7, and an aqueous vehicle for reconstituting the lyophilized formulation.

10. A device for use in delivering a drug by injection, the device comprising a lyophilized formulation according to claim 8, and an aqueous vehicle for reconstituting the lyophilized formulation.

11. A PTH formulation comprising an aqueous vehicle, PTH at a concentration within the range from about 1 mg/mL to about 2 mg/mL and zinc chloride at a concentration in the range from about 20 to about 40 mM.

12. A process for preparing a formulation comprising stabilized PTH, comprising the step of combining PTH, an amount of at least one divalent cation effective to stabilize said PTH, and water.

13. A process for preparing an injectable formulation comprising stabilized PTH, comprising the step of reconstituting, in an aqueous vehicle, a lyophilized preparation comprising PTH and at least one divalent cation in an amount effective to stabilize the reconstituted PTH.

14. A process for preparing an injectable formulation comprising stabilized PTH, comprising the step of reconstituting, in an aqueous vehicle, a lyophilized preparation comprising PTH, wherein said vehicle comprises at least one divalent cation in an amount effective to stabilize the reconstituted PTH.

15. A method of treating osteoporosis comprising the steps of administering to a subject in need thereof a formulation according to claim 1.

16. The method of claim 15, wherein said subject is a human.

17. A method of diagnosing hypoparathyroidism comprising the steps of
(a) administering to a subject in need thereof a PTH formulation according to claim 1; and
(b) determining the effect of the administered PTH on urinary cAMP levels, with cAMP elevation being indicative of a hypoparathyroid condition.

18. The method of claim 17, wherein said subject is a human.
